SEQUENCE LISTING (3 sequences)

SEQ ID NO: 1 - protein, 30 aa, Artificial Sequence
  Inventor Tanihara, Masao Inventor Aoshima, Hisae
  Description of Artificial Sequencepeptide
  MISC_FEATURE (1)..(30): Xaa is defined as 4Hyp
PXGPXGPXGPXGPQGIAGPXGPXGPXGPXG

SEQ ID NO: 2 - protein, 30 aa, Artificial Sequence
  Description of Artificial Sequencepeptide
  MISC_FEATURE (1)..(30): Xaa is defined as 4Hyp
PXGPXGPXGPXGPXGPXGPXGPXGPXGPXG

SEQ ID NO: 3 - protein, 30 aa, Artificial Sequence
  Description of Artificial Sequencepeptide
  misc_feature (1)..(30): Xaa is defined as 4Hyp
PXGPXGPXGPXGPLGIAGPXGPXGPXGPXG

(19) United States
(12) Patent Application Publication
(10) Pub. No.: US 2007/0207955 A1
(43) Pub. Date: Sep. 6, 2007

(54) Novel polypeptide and process for producing the same, and collagenase inhibitor

(76) Inventors: Masao Tanihara, Kyoto-shi (JP); Hisae Aoshima, Kyoto-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/365,936

(22) Filed: Mar. 2, 2006

(51) Int. Cl. A61K 38/16; C07K 14/47

(52) U.S. Cl. 514/12; 530/324

(57) ABSTRACT

The present invention provides a novel biodegradable and biosorbable polypeptide which is free from a risk of an infection by a pathogenic organism or a transmission of a causative factor, or a risk of an undesirable side effect, and a process for producing the same, as well as a collagenase inhibitor comprising the polypeptide and having a high collagenase inhibitory action. The polypeptide contains a peptide unit having an amino acid sequence represented by the following formula (1), and a peptide unit having an amino acid sequence represented by the following formula (2):
-Pro-X-Gly- (1)
-Pro-Y-Gly-Z-Ala-Gly- (2) wherein X represents Pro or Hyp; Y represents Gln, Asn, Leu, Ile, Val or Ala; and Z represents Ile or Leu. The molar ratio of the peptide unit (1) relative to the peptide unit (2) is about 99/1 to 1/99.

### FIELD OF THE INVENTION

The present invention relates to a novel polypeptide having biodegradability and biosorbability (or bioabsorbability), and a process for producing the same, as well as a collagenase inhibitor comprising the novel polypeptide. More specifically, the present invention relates to a novel polypeptide useful for a biomaterial or biocompatible material which is free from a risk of an infection by a pathogenic organism (or a causative factor) or an undesirable side effect, and which has a high safety, and a process for producing the same. Such a biomaterial or biocompatible material includes, for example, a medical material such as a carrier or support for a tissue engineering, a carrier or support for a regenerative medical treatment, a tissue binding agent or an antiadhesive material, a suture for a surgical operation, a hemostatic material and a contact lens; a raw material for a pharmaceutical preparation; a raw material for a cosmetic preparation; and others. Further, the present invention relates to a collagenase inhibitor useful for a biomaterial or a medical material, an active ingredient of a pharmaceutical preparation, a raw material for a cosmetic preparation or an additive for a food composition, and others.

### BACKGROUND OF THE INVENTION

A collagen is a fibrous protein found in all multicellular organisms. The collagen is a main component of skins or bones, and occupies 25% of total proteins in mammals. A typical collagen molecule has a rope-like superhelical structure, which is referred to as a triple helical structure, comprising three collagen polypeptide chains. The collagen is particularly rich in proline (Pro) and glycine (Gly). These two amino acid residues are important to form a stable triple helical structure of the collagen.

As methods for using a collagen as a biomaterial, there may be mentioned, for example, a method of grafting or transplanting an intact or lyophilized skin tissue derived from a pig on a skin area damaged by a burn or scald, a method of removing cellular components from a tissue with enzyme treatment, and a method of using a collagen which is solubilized by a treatment with an acidic solution or an enzyme to reconstitute a desirable form. A common preparation method and a common qualitative method are described in Methods Enzymol., Vol. 82, pp. 33 to 64, 1982.

There are various suggestions to utilize a collagen. For example, Japanese Patent Application Laid-Open No. 027192/1996 (JP-08-027192A) discloses a production process of a collagen derivative for imparting moisture and smoothness to skin, which comprises esterifying and modifying an animal tissue containing a collagen with an alcohol, and extracting the modified collagen, as well as a cosmetic base material using the collagen derivative.

Japanese Patent Application Laid-Open No. 097454/1995 (JP-07-097454A) discloses a production process of a water-soluble crosslinked collagen which shows a high regeneration rate of a triple helical structure after thermal denaturation, and the process comprises subjecting a water-soluble collagen to a crosslinking treatment with a bifunctional alkylene diimidate cross-linker having imide ester groups at both ends of the methylene chain.

Japanese Patent Application Laid-Open No. 053548/1996 (JP-08-053548A) discloses a matrix of a collagen and a synthetic polymer (a collagen-synthetic polymer matrix) which has a low immunogenicity and is useful for preparation of biocompatible implants utilized for various medical applications, and a production process of the matrix comprises reacting a collagen with a first synthetic hydrophilic polymer to form a collagen-synthetic polymer matrix, and further reacting the collagen-synthetic polymer matrix with a reactant such as a second synthetic hydrophilic polymer, a biologically active substance, a glycosaminoglycan and a derivative thereof, a chemical crosslinking agent, an esterifying agent, an amidating agent, an acylating agent, an amino acid, a polypeptide, or others.

Japanese Patent Application Laid-Open No. 278312/1995 (JP-07-278312A) discloses a united material containing a hydrophilic synthetic polymer covalently bonded to a chemically modified collagen which is substantially a nonfiberous form at pH 7. The literature discloses that the united material is particularly useful for opthalmological devices, and optically transparent, and that the united material has a biocompatibility.

Japanese Patent Application Laid-Open No. 000158/1993 (JP-05-000158A) discloses a production process of a collagenic membrane-like substance, which comprises crushing a collagen matrix, centrifuging the crushed matrix under a high centrifugal field, homogenizing the resultant precipitate to obtain a paste, casting the paste, and drying the cast paste at a temperature of not higher than 37° C. The literature also discloses that the collagen membrane-like substance has a biocompatibility and a non-inflammatory property, and is useful for repairing a tissue as an artificial implantation matter.

Japanese Patent Application Laid-Open No. 125100/1993 (JP-05-125100A) discloses a soluble fish scale collagen having high-purity and a production process thereof, and the process comprises pepsinating an intact or deashed fish scale.

Japanese Patent Application Laid-Open No. 228506/1994 (JP-06-228506A) discloses a production process of a dry particulate or powdery soluble collagen, which comprises injecting a collagen solution through a nozzle into 70 to 90% ethanol medium to form a strand-like or membranous product, drying the product, and chopping or grinding the dried product.

Japanese Patent Application Laid-Open No. 276003/1996 (JP-08-276003A) discloses use of an unbaked single-crystal hydroxyapatite as a material for repairing a biological hard tissue (such as a bone), through attaching the single crystal to at least part of a low antigenic collagen fiber.

Japanese Patent Application Laid-Open No. 041425/1996 (JP-08-041425A) discloses a method which comprises removing fragments of cells or tissues from a collagen solution and subjecting the residue to an alkali treatment, for removing prion in a collagen derived from an animal or human being, and discloses a collagen obtained by this method.

Moreover, regarding methods for chemical synthesis of collagen analogues, it has been reported that a soluble polyamide having a molecular weight of 16,000 to 21,000 is obtained by dissolving a p-nitrophenyl ester of a peptide represented by the formula: Pro-Ser-Gly, or a p-nitrophenyl ester of a peptide represented by the formula: Pro-Ala-Gly in dimethylformamide, adding triethylamine thereto, and allowing to stand the mixture for 24 hours (J. Mol. Biol., Vol. 63, pp. 85 to 99, 1972). In this literature, the soluble polyamide is estimated to form a triple helical structure based on the circular dichroism spectra. However, there are not referred to properties of the obtained polymer.

It also has been reported that a method for obtaining a polyamide, which comprises dissolving a 50-mer peptide containing the sequence Val-Pro-Gly-Val-Gly derived from elastin in dimethylsulfoxide, adding 2 equivalents of 1-ethyl-3-(3-dimethylaminopropyl)-carbodiimide, 1 equivalent of 1-hydroxybenzotriazole and 1.6 equivalents of N-methylmorpholine thereto, allowing to stand the mixture for 14 days, and dialyzing the resultant mixture with a dialysis membrane (molecular weight cut-off: 50,000) (Int. J. Peptide Protein Res., Vol. 46, pp. 453 to 463, 1995).

It has been known that a collagenase (Matrix metalloproteinase-1), which is an enzyme for specifically decomposing a collagen, increases an enzymatic activity by an inflammatory reaction, an inflammatory disease or aging, and decomposes a collagen. Therefore, it has been considered that inhibition of the collagenase activity is effective for suppression of the inflammatory reaction, alleviation of various symptoms caused by the inflammatory reaction, antiaging of skin, prevention of osteoporosis, and others.

There have been various suggestions for utilizing substances having a collagenase inhibitory action. For example, Japanese Patent Application Laid-Open No. 192565/2003 (JP-2003-192565A) discloses an antiaging cosmetic preparation containing a plant extract having a collagenase inhibitory action and a fish collagen.

Japanese Patent Application Laid-Open No. 183173/2003 (JP-2003-183A) discloses an external preparation for skin, and food and drink, which comprises a collagenase inhibitor containing an extract from a plant such as Nyctanthes albortristis, Piper chaba, Anthocephalus indicus, or Crotalaria cysoides as an effective (or active) ingredient.

Japanese Patent Application Laid-Open No. 176219/2003 (JP-2003-176219A) discloses an external preparation for skin, which contains an antiseptic and mildewproof agent, and a tripeptide, wherein the tripeptide is a decomposed product of a collagen or gelatin, and has an amino acid sequence represented by the formula: (Gly-X—Y)ₙ (in the formula, Gly represents glycine residue, X and Y are the same or different, each representing an amino acid residue, and “n” denotes a positive integer).

Japanese Patent Application Laid-Open No. 104986/2002 (JP-2002-104986A) discloses an agent for anti-periodontic diseases, which contains a Schizandrae fructus extract as an effective ingredient, and has an action for inhibiting collagenase produced by periodontal disease germ Porphyromonas gingivalis.

Japanese Patent Application Laid-Open No. 247469/2001 (JP-2001-247469A) discloses an agent for anti-caries and anti-periodontic diseases, which contains α- and/or γ-mangosteen extracted from Garcinia mangostana L. and a pericarp thereof as an effective ingredient, and has an action for inhibiting collagenase produced by periodontal disease germ Porphyromonas gingivalis; as well as an oral composition, and a food and drink containing the agent.

Japanese Patent Application Laid-Open No. 241287/1997 (JP-09-241287A) discloses a collagenase inhibitory substance FO-5904, which is a collagenase inhibitory substance collected from a culture of a microorganism belonging to Aspergillus such as Aspergillus niger, and is useful for an antirheumatic, an antiinflammatory agent, an anticancer agent, and a therapeutic agent for influenza viral infection.

Japanese Patent Application Laid-Open No. 235293/1997 (JP-09-235293A) discloses a novel triterpene compound which is extracted from a fungus fruit body such as Daedalea dickinsi, and has a collagenase inhibitory action. This document mentions that the triterpene compound is useful as a therapeutic agent for a disease such as an articular disease, a bone resorption disease, a periodontal disease, a corneal ulcer, or an epidermolysis bullosa.

Japanese Patent Application Laid-Open No. 304770/1995 (JP-07-304770A) discloses a novel benzoazepinone derivative which has a strong collagenase inhibitory action, and is useful as a therapeutic drug for a variety of diseases due to enhancement of a collagenase activity.

Japanese Patent Application Laid-Open No. 291873/1995 (JP-07-291873A) discloses an inhibitor for collagenase activity, which contains a solvent extract of at least one selected from the group consisting of Psidium guajava leaves, Tamarix chinensis lour and Trapa natans, and is useful for a food or pharmaceutical.

Japanese Patent Application Laid-Open No. 163287/1993 (JP-05-163287A) discloses a novel peptide derivative which is used as an inhibitor for a bacterial collagenase belonging to a zinc protein, and has a phosphine chelate group PO₂—CH₂ capable of strongly interacting with a zinc atom in an active site of the collagenase.

Moreover, there have been reported structural characteristics of a collagen-like heterotrimer as a model of a site to be decomposed by a collagenase in a collagen type I (J. Mol. Biol., Vol. 319, pp. 1235-1242, 2002).

On the other hand, as described in the above mentioned JP-08-041425A, a causative substance of sheep tremor or bovine spongiform encephalopathy is an infectious protein called as prion, and the infectious protein is considered as one of causes of human Creutzfeldt-Jakob disease infection. The prion is a protein, and it is indicated that prion is difficult to deactivate with a conventional pasteurization or sterilization method, further that prion is infectious over species (Nature Review, Vol. 2, pp. 118 to 126, 2001).

In general, a natural product and an extract therefrom (e.g., a collagen derived from bovine or pig) is frequently used as a raw material for medical kits (devices) or pharmaceutical preparations, and cosmetic preparations, and foods and drinks. Accordingly, there have been always existed the risk of an infection (or a transmission) to pathogenic organisms or a causative factor such as prion which cannot be removed by conventional pasteurizations or sterilizations.

Moreover, since various cell adhesion sites are found in a naturally occurring collagen, the naturally occurring collagen cannot exert cell selectivity for any applications. For example, in the case using a collagen as a material for inducing a nerval axon, migration or growth rate of surrounding fibroblast is faster than elongation rate of the axon, resulting in forming scarring tissue, and the axon cannot be elongated. It is therefore necessary to take a step to cover around the collagen with a material for protecting migration of fibroblast, or others. On the other hand, synthetic chemicals as described in JP-07-304770A have insufficient biodegradability or biosorbability, and also involve a risk of a side effect, and others.

### SUMMARY OF THE INVENTION

It is therefore an object of the present invention to provide a novel biodegradable polypeptide which is free from a risk of an infection by a pathogenic organism, a risk of a transmission of a causative factor, or a risk of an undesirable side effect, and a process for producing the same.

It is another object of the present invention to provide a novel polypeptide useful as a highly safe biomaterial or biocompatible material, and a process for producing the same.

It is still another object of the present invention to provide a process for efficiently producing a polypeptide having the above-mentioned properties with inhibiting dimerization or cyclization reaction.

It is a further object of the present invention to provide a biodegradable collagenase inhibitor (or a novel polypeptide) which has a high collagenase inhibitory action, and is free from a risk of an infection by a pathogenic organism, a risk of a transmission of a causative factor, or a risk of an undesirable side effect.

A still further object of the present invention is to provide a collagenase inhibitor which has a high safety and is useful as an ingredient of a biomaterial or medical material, a pharmaceutical preparation composition, a cosmetic preparation, and a food composition.

The inventor of the present invention made intensive studies to achieve the above objects and finally found that a collagen-like polypeptide can be produced by condensation of a specific peptide component without cyclization. Also, the inventors of the invention found that the polypeptide has a collagenase inhibitory action. The present invention was accomplished based on the above findings.

That is, the novel polypeptide of the present invention contains a peptide unit having an amino acid sequence represented by the following formula (1), and a peptide unit having an amino acid sequence represented by the following formula (2):
-Pro-X-Gly- (1)
-Pro-Y-Gly-Z-Ala-Gly- (2)

wherein X represents Pro or Hyp; Y represents Gln, Asn, Leu, Ile, Val or Ala; and Z represents Ile or Leu.

The proportion (molar ratio) of the peptide unit (1) relative to the peptide unit (2) may be [(1)/(2)]=about 99/1 to 1/99 (for example, about 99/1 to 30/70).

In the formulae (1) and (2), the polypeptide may satisfy the following condition (i) or (ii) as follows: (i) X is Hyp, Y is Gln, Asn, Leu, Ile, Val or Ala, and Z is Ile or Leu; or (ii) X is Pro, Y is Gln, Asn, Leu, Ile, Val or Ala, and Z is Ile or Leu.

The polypeptide of the present invention is degradable with a collagenase which is a collagen-digesting enzyme, therefore, is also degradable in a living body of a mammal. Accordingly, the polypeptide is biodegradable and biosorbable (that is, the polypeptide has biodegradability and biosorbability). The polypeptide shows positive Cotton effect at a wavelength in a range of 220 to 230 nm and negative Cotton effect at a wavelength in a range of 195 to 205 nm in a circular dichroism spectrum. This fact shows that at least part (part or whole) of the polypeptide forms a triple helical structure. The polypeptide of the present invention may show a peak of the molecular weight in the range from about 5×10² to 500×10⁴ (e.g., about 5×10³ to 500×10⁴) in the molecular weight distribution. Moreover, the polypeptide of the present invention is capable of forming a collagen tissue (collagenous tissue or collagen-like tissue).

In the present invention, the polypeptide may be obtained by condensing an amino acid component and/or peptide fragment component (or peptide component) which at least contains an amino acid or peptide fragment corresponding to the formula (1) and an amino acid or peptide fragment corresponding to the formula (2). The polypeptide may be produced by, for example, (a) condensing a peptide component which at least contains a peptide having the both amino acid sequences represented by the formulae (1) and (2), or (b) condensing a peptide component which at least contains a peptide having an amino acid sequence represented by the formula (1) and a peptide having an amino acid sequence represented by the formula (2).

In the condensation process of the peptide component, the reaction may be usually carried out by condensing the peptide component in the presence of at least a dehydrating and condensing agent (e.g., a carbodiimide-series condensing agent, a fluorophosphate-series condensing agent, and a diphenylphosphorylazide) in a solvent (water and/or an organic solvent). Moreover, the reaction may be carried out in the presence of both the dehydrating and condensing agent and a condensing auxiliary (or dehydrating auxiliary) [for example, an N-hydroxypolycarboxylic acid imide, an N-hydroxytriazole (e.g., an N-hydroxybenzotriazole such as 1-hydroxybenzotriazole), a triazine, and ethyl ester of 2-hydroxyimino-2-cyanoacetic acid]. In the case of conducting the reaction in a non-aqueous solvent (a solvent free from water), the proportion of the dehydrating and condensing agent may be about 0.7 to 5 mol relative to 1 mol of the total amount of the amino acid or peptide component. In the case of conducting the reaction in an aqueous solvent (a solvent containing water), the proportion of the dehydrating and condensing agent may be about 2 to 500 mol relative to 1 mol of the total amount of the amino acid or peptide component. The proportion of the condensing auxiliary may be about 0.5 to 5 mol relative to 1 mol of the total amount of the peptide component.

The novel polypeptide may be used for inhibiting a collagenase activity. The present invention also includes a collagenase inhibitor comprising the polypeptide. The polypeptide or collagenase inhibitor contains an amino acid sequence to be recognized by a collagenase which is a collagen-digesting enzyme. Therefore, the polypeptide is bonded to a collagenase, or decomposed by a collagenase, or bonded to a collagenase and decomposed by the collagenase. Due to such a behavior, the polypeptide can inhibit a collagenase action. Moreover, since the polypeptide is a collagen-like polypeptide having an amide bond, the polypeptide is degradable in a living body of a mammal. That is, the polypeptide is degradable and sorbable (or absorbable) in a living body (that is, the polypeptide has biodegradability and biosorbability (or bioabsorbability)). The collagenase inhibitor may be used, for example, as an ingredient of a biomaterial or medical material, a pharmaceutical preparation composition, a cosmetic preparation, a food composition, and others.

The present invention also includes a cosmetic preparation and a food composition, which contain the polypeptide and inhibit a collagenase activity. Moreover, the present invention further includes a method for inhibiting a collagenase activity, which comprises acting the polypeptide on a collagenase.

### DETAILED DESCRIPTION OF THE INVENTION

In the present invention, amino acid residues are abbreviated to the following condensation codes.

Ala: L-alanine residue

Arg: L-arginine residue

Asn: L-asparagine residue

Asp: L-aspartic acid residue

Cys: L-cysteine residue

Gln: L-glutamine residue

Glu: L-glutamic acid residue

Gly: glycin residue

His: L-histidine residue

Hyp: L-hydroxyproline residue

Ile: L-isoleucine residue

Leu: L-leucine residue

Lys: L-lysine residue

Met: L-methionine residue

Phe: L-phenylalanine residue

Pro: L-proline residue

Sar: sarcosine residue

Ser: L-serine residue

Thr: L-threonine residue

Trp: L-tryptophan residue

Tyr: L-tyrosine residue

Val: L-valine residue

Moreover, in this specification, amino acid sequences of peptide chains are represented in accordance with the conventional expression that N-terminus and C-terminus in an amino acid residue are drawn at the left and the right sides, respectively.

It is essential that the novel polypeptide of the present invention contains a peptide unit having an amino acid sequence represented by the formula: -Pro-X-Gly-. The sequence represented by the formula: -Pro-X-Gly- contributes to stability of the triple helical structure. Therefore, in the case where the proportion of the sequence is low, the polypeptide deteriorates in stability of the triple helical structure. Further, this unit may form a repeating structure (oligo- or polypeptide unit structure) represented by the formula: -(Pro-X-Gly)ₙ- in a polypeptide from the viewpoint of stability of a triple helical structure thereof. The repeating number “n” of the sequence is, for example, about 1 to 5000, and preferably about 2 to 3000. The residue “X” may be either Pro or Hyp. In view of stability of the triple helical structure, the residue “X” is more preferably Hyp. Incidentally, Hyp is usually 4Hyp (e.g., trans-4-hydroxy-L-proline) residue.

Moreover, it is essential that the polypeptide of the present invention contains a peptide unit having an amino acid sequence represented by the formula: -Pro-Y-Gly-Z-Ala-Gly-. In the case where the polypeptide does not contain this sequence or the amount of this sequence is too small, degradability (decomposition action) due to a collagenase or inhibitory action to a collagenase is reduced. On the other hand, the amount of the sequence is too large, stability of the triple helical structure is deteriorated. The residue “Y” may be Gln, Asn, Leu, Ile, Val or Ala, and is preferably Gln, Asn, Leu, Val, or Ala. In particular, Gln or Leu is more preferred. The residue “Z” may be either Ile or Leu, and Ile is more preferred. With respect to the combination of the residues “Y” and “Z”, for example, the polypeptide may include a peptide in which the residue “Y” is Gln, Asn, Leu, Ile, Val or Ala (e.g., Gln or Leu), and the residue “Z” is Ile, and a peptide in which the residue “Y” is Gln, Asn, Leu, Ile, Val or Ala (e.g., Gln or Leu), and the residue “Z” is Leu, and others.

With respect to the combination of the residues “X”, “Y” and “Z”, the polypeptide may include a peptide in which the residue “X” is Hyp, the residue “Y” is Gln, Asn, Leu, Ile, Val or Ala (e.g., Gln or Leu), and the residue “Z” is Ile or Leu, a peptide in which the residue “X” is Pro, the residue “Y” is Gln, Asn, Leu, Ile, Val or Ala (e.g., Gln or Leu), and the residue “Z” is Ile or Leu, and others.

Further, unless the physical and biological properties of the obtained polypeptide are deteriorated, the polypeptide of the present invention may contain other amino acid residue or peptide residue (unit). In order that the polypeptide of the present invention exerts useful physical and biological properties, for example, the polypeptide often has at least one amino acid residue or peptide residue selected from the group consisting of Gly, Sar, Ser, Glu, Asp, Lys, H is, Ala, Val, Leu, Arg, Pro, Tyr, and Ile, particularly at least one amino acid residue or peptide residue selected from the group consisting of Gly, Sar, Ser, Glu, Asp, Lys, Arg, Pro, and Val. More specifically, for example, it is preferred to contain an amino acid residue or peptide residue represented by Gly, Sar, Ser, Glu, Asp, Lys, Arg-Gly-Asp, Tyr-Ile-Gly-Ser-Arg, Ile-Lys-Val-Ala-Val, Val-Pro-Gly-Val-Gly, Asp-Gly-Glu-Ala, Gly-Ile-Ala-Gly, His-Ala-Val, Glu-Arg-Leu-Glu, Lys-Asp-Pro-Lys-Arg-Leu, or Arg-Ser-Arg-Lys.

The polypeptide of the present invention may be a physiologically or pharmacologically acceptable salt, and for example, may be a salt with a salifiable compound such as an inorganic acid (e.g., a hydrochloric acid, a sulfuric acid, and a phosphoric acid), an organic acid (e.g., acetic acid, trifluoroacetic acid, lactic acid, tartaric acid, maleic acid, fumaric acid, oxalic acid, malic acid, citric acid, oleic acid, and palmitic acid), a metal (e.g., an alkali metal such as sodium or potassium, an alkaline earth metal such as calcium, and aluminum), or an organic base (e.g., trimethylamine, triethylamine, t-butylamine, benzylamine, diethanolamine, dicyclohexylamine, and arginine). These salifiable compounds may be used singly or in combination. These salts may be obtained by a conventional salt-forming reaction.

In the polypeptide of the present invention, the proportion (molar ratio) of the peptide unit (1) relative to the peptide unit (2) may be selected from the range of [(1)/(2)]=about 99/1 to 1/99 (e.g., about 98/2 to 2/98, and preferably about 95/5 to 5/95), and may be preferably about 99/1 to 30/70 (e.g., about 98/2 to 40/60) and more preferably about 95/5 to 50/50 (e.g., about 95/5 to 70/30).

The proportion (molar ratio) of the total amount of the peptide units (1) and (2) relative to other peptide unit(s) [the former/the latter] may be about 100/0 to 50/50, preferably about 100/0 to 60/40, and more preferably about 100/0 to 70/30.

Such a polypeptide takes a linear polypeptide formation without forming a ring such as a six-membered ring by cyclization, and is soluble in a solvent (for example, water, a hydrophilic solvent such as a sulfoxide (such as dimethyl sulfoxide), dimethylformamide, dimethylacetamide or N-methylpyrrolidone, or a mixed solvent thereof). The polypeptide of the present invention shows, for example, a peak of the molecular weight in the range from about 5×10² to 500×10⁴ (e.g., about 5×10³ to 500×10⁴), preferably about 1×10³ to 300×10⁴ (e.g., about 1×10⁴ to 300×10⁴), preferably about 3×10³ to 200×10⁴ (e.g., about 3×10⁴ to 200×10⁴), and more preferably about 5×10³ to 100×10⁴ (e.g., about 5×10⁴ to 100×10⁴). Incidentally, the molecular weight (or the peak of the molecular weight) of the polypeptide is determined in terms of a globular protein by means of an aqueous gel permeation chromatography (GPC).

Further, the polypeptide of the present invention shows positive Cotton effect at a wavelength in a range of 220 to 230 nm and negative Cotton effect at a wavelength in a range of 195 to 205 nm in circular dichroism spectra. At least one part (that is, part or whole) of the polypeptide is, accordingly, capable of forming a triple helical structure, and the polypeptide forms a collagenous (collagen-like) structure. Incidentally, Cotton effect means a phenomenon caused by difference between an absorption coefficient relative to a right circularly polarized light and that relative to a left at a specific wavelength in an optical rotatory substance.

The polypeptide of the present invention is capable of forming a collagen tissue (or a collagenous tissue). The polypeptide chains having the above-mentioned triple helical structure can self-assemble to form a fibril having a length of several nanometers to several tens nanometers. Further, these fibrils can be arranged to form a fiber structure having a length of several nanometers to several tens nanometers. These can be observed by a transmission electron microscope, a scanning electron microscope, or an atomic force microscope.

The novel polypeptide of the present invention may be obtained by a conventional method which comprises subjecting an amino acid or a peptide fragment (or segment) to a condensation reaction, and is not particularly limited to a specific one as long as the polypeptide finally contains the peptide units (1) and (2). For example, the polypeptide may be obtained by utilizing a condensation reaction between constituent amino acids (or amino acid components), or a condensation reaction between a peptide fragment (or a peptide component) and an amino acid (or an amino acid component). The polypeptide is preferably obtained by a method which comprises preparing a peptide having amino acid sequence(s) represented by the formula (1) and/or (2) in advance, and condensing a peptide component containing the prepared peptide(s).

In the method which comprises condensing the peptide component prepared in advance, the peptide chain of the peptide component can be synthesized in accordance with a conventional peptide synthesis method. Peptides may, for example, be prepared based on a solid-phase synthesis method or a liquid-phase synthesis method, and the solid-phase synthesis method is operationally convenient [for example, see “Zoku Seikagaku Jikken Kouza 2, Tanpakushitsu no Kagaku (Supplemental Handbook of Biochemical Experiments, Chemistry of Protein) (the second volume)” edited by The Japanese Biochemical Society (issued by Tokyo Kagaku Dozin Co., Ltd., May 20, 1987), pp. 641 to 694]. For the peptide synthesis, a conventional manner may be utilized, and the manner may include, for example, a coupling method using a condensing agent, an active esterification method (e.g., a phenyl ester such as p-nitrophenyl ester (ONp) and pentafluorophenyl ester (Opfp), an N-hydroxydicarboxylic imide ester such as N-hydroxysuccinimide ester (ONSu), and 1-hydroxybenzotriazole ester (Obt)), a mixed acid anhydride method, an azide method, and others. In the preferred manner, at least a condensing agent (preferably a condensing agent as mentioned below, in particular a combination of a condensing agent as mentioned below with a condensing auxiliary as mentioned below) may be practically used.

Furthermore, in the peptide synthesis, protection of an amino group, a carboxyl group, and other functional group (e.g., a guanidino group, an imidazolyl group, a mercapto group, a hydroxyl group, and an ω-carboxyl group) with a protective group, and elimination or removal of the protective group with a catalytic reduction or a strong acid treatment (e.g., anhydrous hydrogen fluoride, trifluoromethanesulfonic acid, and trifluoroacetic acid) are repeatedly conducted depending on a species of amino acids or peptide segments. For example, as a protective group for an amino group, there may be utilized benzyloxycarbonyl group (Z), p-methoxybenzyloxycarbonyl group (Z(OMe)), 9-fluorenylmethoxycarbonyl group (Fmoc), t-butoxycarbonyl group (Boc), 3-nitro-2-pyridinesulfenyl group (Npys), and the other groups. As a protective group for a carboxyl group, there may be utilized benzyloxy group (OBzl), phenacyloxy group (OPac), t-butoxy group (OBu), methoxy group (OMe), ethoxy group (OEt), and the other groups. Incidentally, an automatic synthesis apparatus may be utilized for the peptide synthesis.

More specifically, the preparation of the peptide chain with the solid-phase synthesis method may be carried out in accordance with a conventional manner. As a solid-phase resin (or a carrier), there may be utilized a polymer insoluble to a reaction solvent, for example, a styrene-divinylbenzene copolymer (e.g., a chloromethylated resin, a hydroxymethyl resin, a hydroxymethylphenylacetamidemethyl resin, and a 4-methylbenzhydrylamine resin).

In the solid-phase synthesis method, a peptide can be usually produced by the following steps: a step forming a peptide chain corresponding to an objective peptide, which comprises operations (i) to (iii) mentioned below, and a step comprising (iv) detaching the peptide chain from the polymer (resin) and eliminating the protective group(s) from the protected functional group(s) to obtain the objective peptide, and purifying the resulting peptide. The peptide chain-forming step comprises (i) bonding an amino acid or peptide fragment to the above polymer (resin) from C-terminal to N-terminal of the objective peptide, in which the amino acid or peptide fragment has a free α-COOH group and a functional group(s) (e.g., at least an α-amino group of the N-terminal) protected with a protective group(s), (ii) eliminating the protective group from the α-amino group for forming a peptide bond among the bonded amino acid or peptide fragment, and (iii) sequentially repeating the above bonding operation and the eliminating operation to elongate the peptide chain for the formation of the object peptide. In the operation (i) for bonding the amino acid or peptide fragment, an amino acid which is corresponding to the C-terminal of the peptide chain and has a free α-COOH group, and in which at least the N-terminal is protected with a protective group (for example, an Fmoc-amino acid, a Boc-amino acid) is used. Incidentally, from the viewpoint of inhibiting a side reaction, detachment of the peptide chain from the polymer is preferably carried out concurrently with elimination of the protective group with the use of trifluoroacetic acid. Moreover, the resulting peptide may be purified by utilizing a separation and purification means (e.g., a reversed phase liquid chromatography, and a gel-permeation chromatography).

A method for condensing the peptide component containing the peptide synthesized by such a manner may include (a) a method which comprises condensing a peptide component at least containing a peptide having both amino acid sequences represented by the formulae (1) and (2), and (b) a method which comprises condensing a peptide component at least containing a peptide having an amino acid sequence represented by the formula (1) and a peptide having an amino acid sequence represented by the formula (2).

In the former method (a), the peptide having both amino acid sequences represented by the formulae (1) and (2) (that is, a peptide containing both a peptide unit having an amino acid sequence represented by the formula (1) and a peptide unit having an amino acid sequence represented by the formula (2)) may be used singly or in combination. Moreover, in the method (a), as the peptide component, other peptide(s) may be used in addition to the peptide containing the both of the above units, depending on an object polypeptide. Other peptide(s) may include, for example, a peptide containing the above-mentioned other amino acid residue(s) or peptide residue(s). These other peptides may be also used singly or in combination. Incidentally, in the method (a), the proportion of the unit (1) relative to the unit (2) may be easily adjusted by co-condensing a peptide having an amino acid sequence represented by the formula (1) or (2).

Also in the latter method (b), a peptide (oligo- or polypeptide unit) having an amino acid sequence represented by the formula (1), and a peptide having an amino acid sequence represented by the formula (2) may be used singly or in combination. Moreover, in the method (b), as the peptide component, other peptide(s) (e.g., a peptide containing the above-mentioned other amino acid residue or peptide residue) may be used in addition to these peptides (1) and (2), depending on an object polypeptide. These other peptides may be also used singly or in combination.

The condensation reaction of these peptides is usually carried out in a solvent. The solvent may be capable of dissolving or suspending (partly or wholly dissolving) the peptide components and the compound, and there may be usually employed water and/or an organic solvent. Examples of the solvent may include water, an amide (e.g., dimethylformamide, dimethylacetamide, and hexamethylphosphoramide), a sulfoxide (e.g., dimethylsulfoxide), a nitrogen-containing cyclic compound (e.g., N-methylpyrrolidone, and pyridine), a nitrile (e.g., acetonitrile), an ether (e.g., dioxane, and tetrahydrofuran), an alcohol (e.g., methyl alcohol, ethyl alcohol, and propyl alcohol), and a mixed solvent thereof. Among these solvents, water, dimethylformamide, or dimethylsulfoxide is practically used.

The reaction of these peptides may be usually carried out in the presence of at least a dehydrating agent (a dehydrating and condensing agent) or a condensing agent. The reaction with these peptide components in the presence of a dehydrating and condensing agent and a condensing auxiliary (synergist) smoothly produces a polypeptide with inhibiting dimerization or cyclization.

The dehydrating and condensing agent is not particularly limited to a specific one as far as the agent can conduct dehydration and condensation efficiently in the above-mentioned solvent. For example, the dehydrating and condensing agent (the dehydrating agent) may include a carbodiimide-series condensing agent [e.g., diisopropylcarbodiimide (DIPC), 1-ethyl-3-(3-dimethylaminopropyl)-carbodiimide (EDC=WSCI), 1-ethyl-3-(3-dimethylaminopropyl)-carbodiimide hydrochloride (WSCI.HCl), and dicyclohexylcarbodiimide (DCC)], a fluorophosphate-series condensing agent [e.g., O-(7-azabenzotriazol-1-yl)-1,1,3,3-tetramethyluronium hexafluorophosphate, O-benzotriazol-1-yl-N,N,N′,N′-tetramethyluronium hexafluorophosphate, benzotriazol-1-yl-oxy-tris-pyrrolidinophosphonium hexafluorophosphate, and a salt of benzotriazol-1-yl-tris(dimethylamino)phosphonium hexafluorophosphide (BOP)], diphenylphosphorylazide (DPPA), and others. The dehydrating and condensing agent(s) may be used singly, or used as a mixture in combination thereof. The preferred dehydrating and condensing agent includes a carbodiimide-series condensing agent [e.g., 1-ethyl-3-(3-dimethylaminopropyl)-carbodiimide, and 1-ethyl-3-(3-dimethylaminopropyl)-carbodiimide hydrochloride].

The condensing auxiliary is not particularly limited to a specific one as long as the condensing auxiliary can facilitate the reaction of the condensing agent. For example, there may be mentioned an N-hydroxypolycarboxylic imide [e.g., an N-hydroxydicarboxylic imide such as N-hydroxysuccinic imide (HONSu) or N-hydroxy-5-norbornene-2,3-dicarboxylicimide (HONB)]; an N-hydroxytriazole [e.g., an N-hydroxybenzotriazole such as 1-hydroxybenzotriazole (HOBt)]; a triazine such as 3-hydroxy-4-oxo-3,4-dihydro-1,2,3-benzotriazine (HOObt); ethyl ester of 2-hydroxyimino-2-cyanoacetic acid; and others. These condensing auxiliaries may be also used singly or in combination. The preferred condensing auxiliary includes an N-hydroxydicarboxylic imide [e.g., HONSu], an N-hydroxybenzotriazole or N-hydroxybenzotriazine [e.g., HOBt].

The dehydrating and condensing agent may be suitably used in combination with the condensing auxiliary. As a combination of the dehydrating and condensing agent with the condensing auxiliary, there may be mentioned, for example, DCC-HONSu (HOBt or HOObt), WSCI-HONSu (HOBt or HOObt), and other combinations.

The amount of the dehydrating and condensing agent is, in a water-free solvent system, usually about 0.7 to 5 mol, preferably about 0.8 to 2.5 mol, and more preferably about 0.9 to 2.3 mol (e.g., about 1 to 2 mol) relative to 1 mol of the total molar amount of the peptides. In a water-containing solvent (or an aqueous solvent) system, since the dehydrating and condensing agent may be deactivated by water, the amount of the dehydrating and condensing agent is usually about 2 to 500 mol (e.g., about 2 to 50 mol), preferably about 5 to 250 mol (e.g., about 5 to 25 mol), and more preferably about 10 to 125 mol (e.g., about 10 to 20 mol) relative to 1 mol of a total molar amount of the peptides. The amount of the condensing auxiliary is, for example, about 0.5 to 5 mol, preferably about 0.7 to 2 mol, and more preferably about 0.8 to 1.5 mol relative to 1 mol of a total molar amount of the peptides irrespective of a kind or species of the solvent.

In the condensation reaction of the present invention, the pH of the reaction system may be adjusted, or a base being inert for the reaction may be added to the system. The pH may be usually adjusted with an inorganic base [e.g., sodium hydroxide, potassium hydroxide, sodium carbonate, and sodium hydrogen carbonate], an organic base, an inorganic acid [e.g., hydrochloric acid], or an organic acid. The pH of the reaction mixture is usually adjusted to approximately neutral pH (pH=about 6 to 8). As the base being inert for the reaction, there may be exemplified a tertiary amine (e.g., a trialkylamine such as trimethylamine, triethylamine or diisopropylethylamine, and a heterocyclic tertiary amine such as N-methylmorpholine or pyridine), and others. The amount of the base may be usually selected within a range from one to two times as much as the total molar amount of the peptides.

The formation of a triple helical structure in the polypeptide of the present invention can be usually proved by measuring circular dichroism spectra for a solution of the polypeptide. In particular, regarding circular dichroism spectra, it has been reported that a naturally-occurring collagen and peptide chain forming a triple helical structure distinctively shows positive Cotton effect at a wavelength in range of 220 to 230 nm and negative Cotton effect at a wavelength in range of 195 to 205 nm (J. Mol. Biol., Vol. 63 pp. 85 to 99, 1972).

Moreover, since the polypeptide is a collagen-like polypeptide capable of forming a collagen tissue, the polypeptide is free from a risk of an infection of a pathogenic organism or a transmission of a causative factor [for example, a protein converted into a pathological protein (e.g., abnormal prion)], and has a high safety. Further, since the polypeptide is degradable and sorbable in a living body (or is biodegradable and biosorbable), there is no possibility of an undesirable side effect. Furthermore, the polypeptide is also excellent in cytophilicity or biocompatibility. Therefore, the polypeptide is useful for a biomaterial or a biocompatible material, for example, an artificial collagen. The polypeptide of the present invention is applicable to a tissue (e.g., an epidermal tissue, and a dermal tissue) of a subject. The subject may include human beings, and nonhumans (such as monkeys, sheep, bovines, horses, dogs, cats, rabbits, rats, and mice). Moreover, the polypeptide of the present invention may be used for avoiding or preventing against an infection (or a transmission) originated from (caused by) a polypeptide (e.g., an infection or a transmission of a pathogenic organism or a causative factor present in a polypeptide). Accordingly, the polypeptide of the present invention may be effectively utilized in a damaged area [for example, a diseased area or an injured area (e.g., an area damaged by a scratch or a burn or scald)] or a dissected (or an incised) area [for example, a dissected area such as a surgical cut]. For example, the polypeptide of the present invention is utilized as a medical material such as a carrier for a tissue engineering, a carrier for a regenerative medical treatment (e.g., an artificial skin), a tissue binding agent or an antiadhesive material, a suture for a surgical operation, a hemostatic material and a contact lens; a raw material (or base material) for a pharmaceutical preparation; a raw material (or base material) for a cosmetic preparation; a food additive; and others.

The polypeptide of the present invention can be shaped or molded by a conventional manner depending on various applications. The form to be used of the polypeptide may be, therefore, a liquid form (e.g., a solution, and a suspension), a particulate form, a two-dimensional form (e.g., a film, and a sheet), or a three-dimensional form. For example, a sheet or film of the polypeptide may be obtained by casting a solution or suspension of the polypeptide on a releasable substrate (support) (e.g., a sheet made from a fluorine-containing resin (polytetrafluoroethylene)) and drying the cast substrate. Moreover, a fibrous substance is obtained by extruding a solution or suspension of the polypeptide through a nozzle in a solution containing a salt of high concentration or in a solvent incapable of dissolving the polypeptide. Further, a gelatinous substance may be obtained by allowing to stand an aqueous solution or suspension of the polypeptide, or if necessary, with adding a polyvalent crosslinkable reagent (e.g., glutaraldehyde) thereto. Further, a sponge-like porous substance may be obtained by lyophilizing the resultant gelatinous substance. Furthermore, a porous substance can be also obtained by stirring the aqueous solution or suspension of the polypeptide to foam, and drying.

Further, the polypeptide of the present invention may be used as a coating agent. For example, a surface of a substrate may be coated with the polypeptide of the present invention by coating or spraying the surface with the solution or suspension of the polypeptide and drying the coated or sprayed layer. The substrate may be a shaped article made of various materials such as a metal, a ceramic, a plastic, a natural polymer, or the like. The form of the shaped article may be a two-dimensional structure or a three-dimensional structure, e.g., a particulate form, a linear or fibrous form, a film or sheet form. Further, the polypeptide may be held or supported by impregnating a porous substance into the solution or suspension of the polypeptide. The porous substance may include, for example, a particulate porous substance, a two-dimensional porous substance such as a paper made from a cellulose fiber (cellulosic paper), a nonwoven or woven fabric, and a three-dimensional porous substance such as a cylindrical substance.

Moreover, the polypeptide of the present invention contains an amino acid sequence recognized by a collagenase, binds to a collagenase, and is decomposed by a collagenase. Thus, since the polypeptide has a high bonding action to a collagenase, that is, a high inhibitory action to the collagenase activity, the polypeptide can be used for a collagenase inhibitor. Further, the collagenase inhibitor of the present invention is high in safety, and is excellent in biocompatibility. Therefore, the collagenase inhibitor of the present invention may be used for various applications regarding a subject which requires inhibition of a collagenase activity. As such a subject, there may be mentioned, for example, human beings, and in addition, nonhumans such as monkeys, sheep, bovines, horses, dogs, cats, rabbits, rats, and mice. For example, the collagenase inhibitor may be utilized as a component (e.g., a substrate, an effective ingredient, and an additive) contained in a material or composition such as a biomaterial, a medical material, a pharmaceutical preparation composition, a cosmetic preparation, a food composition, and others. In particular, the collagenase inhibitor of the present invention is useful because the inhibitor is safely applied to an affected area having an increased collagenase activity (e.g., an inflammatory site, a joint, a carious site, a periodontal site, or a biological tissue such as a bone or a cornea).

In the biomaterial or the medical material, the collagenase inhibitor may be used as an additive. Moreover, since the collagenase inhibitor is excellent in film-formability or moldability (or formability) and is easy to form a desired shape, the biomaterial or medical material may comprise the collagenase inhibitor alone. For example, the biomaterial may be used in various forms comprising the collagenase inhibitor, e.g., in a liquid form (e.g., a solution or a suspension), a particulate form, a two-dimensional form (e.g., a film or a sheet), and a three-dimensional form. The biomaterial or medical material (or medical supply) may include, for example, a coating agent (or a covering agent) or an endermic liniment, an implant, a hemostatic material, an antiadhesive material, an adhesive material, a tubular material, and a membrane material.

In the case of using the collagenase inhibitor as an additive for the biomaterial or the medical material, the proportion of the collagenase inhibitor may be, for example, about 0.1 to 500 parts by weight, preferably about 1 to 300 parts by weight, and more preferably 5 to 200 parts by weight relative to 100 parts by weight of the substrate.

The pharmaceutical preparation composition may contain at least the collagenase inhibitor. The pharmaceutical preparation composition may be any of a solid pharmaceutical preparation, a semisolid pharmaceutical preparation, or a liquid pharmaceutical preparation. The pharmaceutical preparation composition may be either a pharmaceutical preparation or a quasi drug, and may be utilized as preparations of a variety of dosage forms. Examples of the solid pharmaceutical preparation may include powders, granules, tablets, lozenges, gumis, pills, and capsules. Examples of the semisolid pharmaceutical preparation may include ointments (including creams, or eye ointments), cataplasms, plasters and pressure sensitive adhesives, and suppositories. Examples of the liquid pharmaceutical preparation may include aerosols, suspensions, emulsions, injectable solutions, ophthalmic solutions, lotions, and liniments. The proportion of the collagenase inhibitor relative to the whole pharmaceutical preparation may be about 0.001 to 99% by weight, preferably about 0.01 to 95% by weight, and more preferably about 0.1 to 90% by weight.

The cosmetic preparation may contain at least the collagenase inhibitor. The cosmetic preparation may be any of a powdery cosmetic preparation containing a powdery base material, a solid or semisolid cosmetic preparation containing a solid or semisolid base material (e.g., an aqueous base material, a gel base material, or an oil-based base material), or a cosmetic preparation liquid containing a liquid base material (an aqueous or oil-based base material). Moreover, the cosmetic preparation usually contains a base material (or a carrier), an active ingredient (e.g., a moisturizing agent) and an additive. The collagenase inhibitor may be contained as at least one component among these components.

The proportion of the collagenase inhibitor may be selected from a wide range depending on the species or dosage form of the cosmetic preparation, for example, selected from the range of about 0.001 to 99% by weight. The proportion of the collagenase inhibitor may be, in the case of using the collagenase inhibitor as a base material, about 10 to 99% by weight and preferably about 20 to 99% by weight relative to the whole cosmetic preparation. In the case of using the collagenase inhibitor as an active ingredient, the proportion of the collagenase inhibitor may be, for example, about 0.001 to 95% by weight and preferably about 0.01 to 90% by weight relative to the whole cosmetic preparation. Moreover, in the case of the collagenase inhibitor as an additive, the proportion of the collagenase inhibitor may be about 0.001 to 40% by weight and preferably about 0.01 to 30% by weight relative to the whole cosmetic preparation.

The food composition may contain at least the collagenase inhibitor. The food composition may be any of a powdery composition containing a powdery base material, a solid or semisolid composition containing a solid or semisolid base material, a liquid composition containing a liquid base material, or a mixture thereof. The food composition usually contains a base material (or a carrier), an active ingredient, and an additive (e.g., a food additive, and a seasoning). The collagenase inhibitor may be contained as at least one component among these components. The food composition is useful for various foods, and in addition, functional foods, e.g., a health food, a health supplement, a functional food, a food for specified uses, and a food with health claims. Moreover, the food composition of the present invention may include animal feeding stuffs for domestic animals (e.g., cattle, pigs, and sheep), pet animals (e.g., mammals such as dogs and cats, birds (or poultry), and reptiles), fish (e.g., cultured fish such as sea breams and eels; and aquarium fishes such as goldfishes), and experimental (or laboratory) animals (e.g., rats).

The proportion of the collagenase inhibitor may be selected from a wide range depending on the species or form of the food composition, for example, selected from the range of about 0.001 to 99% by weight. The proportion of the collagenase inhibitor may be, in the case of using the collagenase inhibitor as a base material, about 10 to 90% by weight and preferably about 20 to 80% by weight relative to the whole food composition. In the case of using the collagenase inhibitor as an additive, the proportion of the collagenase inhibitor may be about 0.001 to 40% by weight and preferably about 0.01 to 30% by weight relative to the whole food composition. Moreover, in the case of using the collagenase inhibitor as an active ingredient, the proportion of the collagenase inhibitor may be, for example, about 0.001 to 90% by weight and preferably 0.01 to 80% by weight relative to the whole food composition.

The polypeptide or collagenase inhibitor of the present invention may be used with sterilization or pasteurization. In particular, in the case of using the polypeptide or collagenase inhibitor for a medical purpose, the polypeptide or collagenase inhibitor is preferably sterilized or pasteurized. A sterilization or pasteurization method may include various methods, for example, pasteurization with steam such as a heated and damp steam, pasteurization with a gamma ray, pasteurization with ethylene oxide gas, sterilization with a pharmaceutical preparation, sterilization with an ultraviolet ray, and others. Among these methods, pasteurization with a gamma ray and pasteurization with ethylene oxide gas are preferred from the viewpoint of pasteurization efficiency and low (or light) adverse effects on a material to be used.

The polypeptide of the present invention is free from a risk of an infection by a pathogenic organism or a risk of a side effect, and is excellent in cytophilicity or biocompatibility with high safety. In addition, since the polypeptide is highly degradable in a living body, it is also expected that the polypeptide has an excellent sorbability (or absorbability) in a living body. Therefore, the polypeptide is suitable for a base material (or a substrate) of a material (or member) for implant in a living body. Further, the polypeptide can be produced by a simple method that involves a condensation reaction with inhibiting dimerization or cyclization reaction. Furthermore, the polypeptide or collagenase inhibitor of the present invention has a high collagenase inhibitory action, and is degradable in a living body without a risk of an infection by a pathogenic organism or a risk of a side effect. Therefore, the polypeptide or collagenase inhibitor is excellent in biocompatibility, has a high safety, and is also useful as an ingredient for a biomaterial or a medical material, a pharmaceutical preparation composition, a cosmetic preparation or a food composition.

The present invention is utilized for a medical material such as a carrier or support for a tissue engineering, a carrier or support (e.g., an artificial skin) for a regenerative medical treatment, a tissue binding agent or an antiadhesive material, a suture for a surgical operation, a hemostatic material and a contact lens; a raw material (or a base material) for a pharmaceutical preparation; a raw material (or a base material) for a cosmetic preparation; a food additive; a coating agent; and others. Since the polypeptide or collagenase inhibitor of the present invention is free from a risk of an infection by a pathogenic organism (or a causative factor) or an undesirable side effect and is degradable in a living body, the polypeptide or collagenase inhibitor is particularly useful for a biomaterial or a medical material, a pharmaceutical preparation composition, a cosmetic preparation, a food composition.

### EXAMPLES

The following examples are intended to describe this invention in further detail and should by no means be interpreted as defining the scope of the invention.

### Example 1

A peptide chain represented by the formula: H-(Pro-Hyp-Gly)₄-Pro-Gln-Gly-Ile-Ala-Gly-(Pro-Hyp-Gly)₄-OH (Sequence ID: 1) was synthesized by a solid-phase synthesis with an automatic peptide synthesis machine. That is, with the use of 0.1 mmol of a particulate resin [HMP glycine, manufactured by Applied Biosystems (US)] which comprised a styrene-divinylbenzene copolymer [molar ratio of styrene relative to divinylbenzene: 99/1] containing 4-(N^{α}-9-(fluorenylmethoxycarbonyl)-glycine)-oxymethyl-phenoxy-methyl group in a proportion of 0.65 mmol/g (resin), the carboxyl terminal of one amino acid was sequentially linked (or bound) to the amino terminal of the other amino acid so as to give an object peptide. In this link reaction, 1 mmol of N^{α}-9-(fluorenylmethoxycarbonyl)-L-proline [Fmoc proline], 1 mmol of N^{α}-9-(fluorenylmethoxycarbonyl)-glycine [Fmoc glycine], 1 mmol of N^{α}-9-(fluorenylmethoxycarbonyl)-N^{γ}-trityl-L-glutamine [Fmoc glutamine], 1 mmol of N^{α}-9-(fluorenylmethoxycarbonyl)-L-isoleucine [Fmoc isoleucine] and 1 mmol of N^{α}-9-(fluorenylmethoxycarbonyl)-L-alanine [Fmoc alanine] (each manufactured by Applied Biosystems (US)), and 1 mmol of N^{α}-9-(fluorenylmethoxycarbonyl)-O-t-butyl-L-hydroxyproline [Fmoc hydroxyproline] (manufactured by Bachem AG) were used as amino acids in each linking step.

The peptide resin obtained by the above-mentioned manner was treated with 10 mL of trifluoroacetic acid containing 5% water for 3 hours. The resulting solution was added to diethyl ether to form a precipitate, and the precipitate was further washed with diethyl ether several times to deprotect the peptide and to eliminate the peptide from the resin. The resulting crude product was purified by a column (PD10 column, manufactured by Amarsham Bioscience K.K.) to give a polypeptide. The purified polypeptide obtained in the foregoing manner was subjected to a column chromatography [“AKTA explorer10XT” manufactured by Amarsham Bioscience K.K., column: “Nova-Pak C18”, manufactured by Millipore Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a single peak was shown at a retention time of 12.4 minutes. The molecular weight of the purified polypeptide was determined as 2681.3 based on FAB method mass spectrum (theoretical value: 2679.9). The proportion of the peptide unit (1) relative to the peptide unit (2) was 8/1 (88.9/11.1) (molar ratio).

The circular dichroism spectrum of the obtained polypeptide was measured, and positive Cotton effect was observed at a wavelength of 224 nm and negative Cotton effect at a wavelength of 198 nm. The results confirmed that the polypeptide formed a triple helical structure.

### Example 2

The polypeptide (2.5 mg (0.0009 mmol)) obtained by Example 1, H-(Pro-Hyp-Gly)₄-Pro-Gln-Gly-Ile-Ala-Gly-(Pro-Hyp-Gly)₄-OH, was suspended in 1 mL of dimethyl sulfoxide, and the mixture was stirred at a room temperature. To the mixture were added 0.12 mg (0.0009 mmol) of diisopropylethylamine, 0.12 mg (0.0009 mmol) of 1-hydroxybenzotriazole, and 0.34 mg (0.0018 mmol) of 1-ethyl-3-(3-dimethylaminopropyl)-carbodiimide hydrochloride, and the resulting mixture was further stirred for 2 days at 20° C. The reaction solution was diluted 3-fold with water, and the diluted solution was dialyzed against water for 3 days for removing a reagent (such as a condensing agent) and an unreacted monomer to give a polypeptide. The proportion of the peptide unit (1) relative to the peptide unit (2) was 8/1 (88.9/11.1) (molar ratio).

The resulting polypeptide was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was recognized in the range from 70000 to 100000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide was measured, and positive Cotton effect was observed at a wavelength of 223 nm and negative Cotton effect at a wavelength of 198 nm. The results confirmed that the polypeptide formed a triple helical structure.

### Example 3

A peptide (1.2 mg (0.00045 mmol)) represented by the formula: H-(Pro-Hyp-Gly)₁₀-OH (Sequence ID: 2; manufactured by Peptide Institute, Inc.) and a peptide (1.2 mg (0.00045 mmol)) represented by the formula: H-(Pro-Hyp-Gly)₄-Pro-Gln-Gly-Ile-Ala-Gly-(Pro-Hyp-Gly)₄-OH (Sequence ID:1) which was obtained in the Example 1 were dissolved in 0.25 mL of 10 mM phosphate buffer solution (pH7.4). To the peptide solution were added 0.12 mg (0.0009 mmol) of 1-hydroxybenzotriazole, and 15.7 mg (0.082 mmol) of 1-ethyl-3-(3-dimethylaminopropyl)-carbodiimide hydrochloride, and the resulting mixture was further stirred for 2 days at 20° C. The reaction solution was diluted 10-fold with water, and the diluted solution was dialyzed against water for 3 days for removing a reagent (such as a condensing agent) and an unreacted monomer to give a polypeptide. The proportion of the peptide unit (1) relative to the peptide unit (2) was 18/1 (94.7/5.3) (molar ratio).

The resulting polypeptide was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was recognized in the range from 140000 to 1000000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide was measured, and positive Cotton effect was observed at a wavelength of 224 nm and negative Cotton effect at a wavelength of 196 nm. The results confirmed that the polypeptide formed a triple helical structure.

### Example 4

A peptide (2.2 mg (0.00081 mmol)) represented by the formula: H-(Pro-Hyp-Gly)₁₀-OH (Sequence ID: 2; manufactured by Peptide Institute, Inc.) and a peptide (0.24 mg (0.00009 mmol)) represented by the formula: H-(Pro-Hyp-Gly)₄-Pro-Gln-Gly-Ile-Ala-Gly-(Pro-Hyp-Gly)₄-OH (Sequence ID: 1) which was obtained in the Example 1 were dissolved in 0.25 mL of 10 mM phosphate buffer solution (pH7.4). To the peptide solution were added 0.12 mg (0.0009 mmol) of 1-hydroxybenzotriazole, and 15.7 mg (0.082 mmol) of 1-ethyl-3-(3-dimethylaminopropyl)-carbodiimide hydrochloride, and the resulting mixture was further stirred for 2 days at 20° C. The reaction solution was diluted 10-fold with water, and the diluted solution was dialyzed against water for 3 days for removing a reagent (such as a condensing agent) and an unreacted monomer to give a polypeptide of the present invention. The proportion of the peptide unit (1) relative to the peptide unit (2) was 98/1 (≈99/1) (molar ratio).

The resulting polypeptide was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was recognized in the range from 140000 to 400000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide was measured, and positive Cotton effect was observed at a wavelength of 224 nm and negative Cotton effect at a wavelength of 197 nm. The results confirmed that the polypeptide formed a triple helical structure.

### Comparative Example 1

A peptide (2.4 mg (0.0009 mmol)) represented by the formula: H-(Pro-Hyp-Gly)₁₀-OH (Sequence ID: 2; manufactured by Peptide Institute, Inc.) was suspended in 1 mL of dimethyl sulfoxide, and the mixture was stirred at a room temperature. To the mixture were added 0.12 mg (0.0009 mmol) of diisopropylethylamine, 0.12 mg (0.0009 mmol) of 1-hydroxybenzotriazole, and 0.34 mg (0.0018 mmol) of 1-ethyl-3-(3-dimethylaminopropyl)-carbodiimide hydrochloride, and the resulting mixture was further stirred for 2 days at 20° C. The reaction solution was diluted 3-fold with water, and the diluted solution was dialyzed against water for 3 days for removing a reagent (such as a condensing agent) and an unreacted monomer to give a polypeptide. The proportion of the peptide unit (1) relative to the peptide unit (2) was 100/0 (molar ratio).

The resulting polypeptide was subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl), and the peak of the molecular weight of the polypeptide was recognized in the range from 140000 to 600000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide was measured, and positive Cotton effect was observed at a wavelength of 224 nm and negative Cotton effect at a wavelength of 196 nm. The results confirmed that the polypeptide formed a triple helical structure.

### Test Example 1

Each of the polypeptides (0.025 mg) obtained in the Examples 2 to 4 and Comparative Example 1 was dissolved in 0.05 mL of a 50 mM Tris/HCl buffer (pH=7.5) containing 50 mM NaCl and 10 mMCaCl₂. Further, to each of the solutions was added 200 ng of collagenase (MMP-1, human rheumatoid synovial fibroblast) which was dissolved in 0.05 mL of a 50 mM Tris/HCl buffer (pH=7.5) containing 50 mM NaCl and 10 mM CaCl₂. The resulting mixture was allowed to stand at 37° C. for 24 hours. Then, 0.1 M HCl aqueous solution (0.01 mL) was added to the mixture to stop the enzyme reaction. The mixture was diluted with 10 mM phosphate buffer (pH 7.4) containing 150 mM NaCl, and subjected to a gel-permeation chromatography (AKTA purifier system, manufactured by Amarsham Bioscience K.K., column: Superose 6 HR GL, flow rate: 0.5 mL/min., eluent: 10 mM phosphate buffer (pH 7.4) containing 150 mM NaCl) to measure a change in the molecular weight distribution.

As a result, the molecular weight peak of the polypeptide of Example 2 was reduced to about 540000 by adding a collagenase, compared with about 1000000 in the case of not adding a collagenase. In the same manner, the polypeptide of Example 3 reduced the molecular weight peak from about 800000 to about 400000, and the polypeptide of Example 4 reduced the molecular weight peak from about 700000 to 300000, by adding a collagenase, respectively. On the contrary, in Comparative Example 1, the molecular weight peak was about 540000 regardless of the existence of the collagenase, and the decrease in the peak was not recognized.

### Example 5

A peptide chain represented by the formula: H-(Pro-Hyp-Gly)₄-Pro-Leu-Gly-Ile-Ala-Gly-(Pro-Hyp-Gly)₄-OH (Sequence ID: 3) was synthesized by a solid-phase synthesis with an automatic peptide synthesis machine. That is, with the use of 0.1 mmol of a particulate resin [HMP glycine, manufactured by Applied Biosystems (US)] which comprised a styrene-divinylbenzene copolymer [molar ratio of styrene relative to divinylbenzene: 99/1] containing 4-(N^{α}-9-(fluorenylmethoxycarbonyl)-glycine)-oxymethyl-phenoxy-methyl group in a proportion of 0.65 mmol/g (resin), the carboxyl terminal of one amino acid was sequentially linked (or bound) to the amino terminal of the other amino acid so as to give an object peptide. In this link reaction, 1 mmol of N^{α}-9-(fluorenylmethoxycarbonyl)-L-proline [Fmoc proline], 1 mmol of N^{α}-9-(fluorenylmethoxycarbonyl)-glycine [Fmoc glycine], 1 mmol of N^{α}-9-(fluorenylmethoxycarbonyl)-L-leucine [Fmoc leucine], 1 mmol of N^{α}-9-(fluorenylmethoxycarbonyl)-L-isoleucine [Fmoc isoleucine] and 1 mmol of N^{α}-9-(fluorenylmethoxycarbonyl)-L-alanine [Fmoc alanine] (each manufactured by Applied Biosystems (US)), and 1 mmol of N^{α}-9-(fluorenylmethoxycarbonyl)-O-t-butyl-L-hydroxyproline [Fmoc hydroxyproline] (manufactured by Bachem AG) were used as amino acids in each linking step.

The peptide resin obtained by the above-mentioned manner was treated with 10 mL of trifluoroacetic acid containing 5% by weight of water for 3 hours. The resulting solution was added to diethyl ether to form a precipitate, and the precipitate was further washed with diethyl ether several times to deprotect the polypeptide and to eliminate the polypeptide from the resin. The resulting crude product was purified by a column (PD10 column, manufactured by Amarsham Bioscience K.K.) to give a polypeptide. The purified polypeptide obtained in the foregoing manner was subjected to a column chromatography [“AKTA explorer10XT” manufactured by Amarsham Bioscience K.K., column: “Nova-Pak C18”, manufactured by Millipore Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a single peak was shown at a retention time of 15 minutes. The molecular weight of the purified polypeptide was determined as 2666.3 based on FAB method mass spectrum (theoretical value: 2664.9).

The circular dichroism spectrum of the obtained polypeptide was measured, and positive Cotton effect was observed at a wavelength of 225 nm and negative Cotton effect at a wavelength of 199 nm. The results confirmed that the polypeptide formed a triple helical structure.

### Example 6

The polypeptide (1.2 mg (0.00045 mmol)) obtained in the Example 5, H-(Pro-Hyp-Gly)₄-Pro-Leu-Gly-Ile-Ala-Gly-(Pro-Hyp-Gly)₄-OH, was dissolved in 0.25 mL of 10 mM phosphate buffer solution (pH7.4). To the peptide solution were added 0.12 mg (0.0009 mmol) of 1-hydroxybenzotriazole, and 15.7 mg (0.082 mmol) of 1-ethyl-3-(3-dimethylaminopropyl)-carbodiimide hydrochloride, and the resulting mixture was further stirred for 2 days at 20° C. The reaction solution was diluted 10-fold with water, and the diluted solution was dialyzed against water for 3 days for removing a reagent (such as a condensing agent) and an unreacted monomer to give a polypeptide. The proportion of the peptide unit (1) relative to the peptide unit (2) was 8/1 (88.9/11.1) (molar ratio).

The resulting polypeptide was subjected to a gel-permeation chromatography (AKTA purifier system, manufactured by Amarsham Bioscience K. K., column: Superose 6 HR GL, flow rate: 0.5 mL/min., eluent: 10 mM phosphate buffer (pH 7.4) containing 150 mM NaCl), and the peak of the molecular weight of the polypeptide was recognized in the range from 80000 to 1000000 in the molecular weight distribution. The molecular weight was calculated with a Gel Filtration HMW Calibration Kit (manufactured by Amarsham Bioscience K.K.) as a reference material.

The circular dichroism spectrum of the obtained polypeptide was measured, and positive Cotton effect was observed at a wavelength of 224 nm and negative Cotton effect at a wavelength of 197 nm. The results confirmed that the polypeptide formed a triple helical structure.

### Comparative Example 2

A peptide represented by the formula: H-(Pro-Hyp-Gly)₁₀-OH (Sequence ID: 2; manufactured by Peptide Institute, Inc.) was used as Comparative Example 2. The proportion of the peptide unit (1) relative to the peptide unit (2) was 1/0 (=100/0) (molar ratio). The circular dichroism spectrum of the obtained polypeptide was measured, and positive Cotton effect was observed at a wavelength of 225 nm and negative Cotton effect at a wavelength of 196 nm. The results confirmed that the polypeptide formed a triple helical structure.

### Test Example 2

Each of the polypeptides (0.025 mg) obtained in the Examples 1 to 6 and Comparative Examples 1 and 2 was dissolved in 0.05 mL of a 50 mM Tris/HCl buffer (pH=7.5) containing 50 mM NaCl and 10 mM CaCl₂. Further, to each of the solutions was added 200 ng of collagenase (MMP-1, human rheumatoid synovial fibroblast) which was dissolved in 0.05 mL of a 50 mM Tris/HCl buffer (pH=7.5) containing 50 mM NaCl and 10 mM CaCl₂, and was added 0.01 mL of 1 mM DMSO solution of Dnp-Pro-Leu-Gly-Ile-Ala-Gly-Arg-NH₂ (manufactured by Peptide Institute, Inc., Dnp represents 2,4-dinitrophenyl group). The resulting mixture was allowed to stand at 37° C. for one hour. Then, 0.2 mL of acetonitrile was added to the mixture, and the resultant was centrifuged at 10,000 rpm for 10 minutes. The obtained supernatant was subjected to a column chromatography [“AKTA explorer10XT” manufactured by Amarsham Bioscience K.K., column: “Nova-Pak C18”, manufactured by Millipore Corporation, 3.9 mmφ×150 mm, mobile phase: a mixed solvent of acetonitrile and water containing 0.05 vol. % of trifluoroacetic acid (concentration of acetonitrile was linearly increased from 5 to 50 vol. % for 30 minutes), flow rate: 1.0 mL/min.], and a peak area of Dnp-Pro-Leu-Gly obtained by decomposition of Dnp-Pro-Leu-Gly-Ile-Ala-Gly-Arg-NH₂ under the action of the collagenase was detected.

Incidentally, as a control, a peak area of the peptide Dnp-Pro-Leu-Gly without addition of any polypeptide was measured. The peak area of the control was considered as 100, and the peak area detected by addition of each of the polypeptides obtained in Examples and Comparative Examples was calculated as a relative value to the control. The results showed that, in the case of adding the polypeptides of Examples 1 to 6, the relative values of the detected peak areas were 5, 3, 12, 22, 4, and 2, respectively, that is, the collagenase activity was inhibited. On the contrary, the results showed that, in the case of adding the polypeptides of Comparative Examples 1 and 2, the relative values of the detected peak areas were 97 and 96, respectively, that is, the inhibition of the collagenase activity was not recognized.

What is claimed is:

1. A novel polypeptide containing
a peptide unit having an amino acid sequence represented by the following formula (1), and
a peptide unit having an amino acid sequence represented by the following formula (2):
-Pro-X-Gly- (1)
-Pro-Y-Gly-Z-Ala-Gly- (2)
wherein X represents Pro or Hyp; Y represents Gln, Asn, Leu, Ile, Val or Ala; and Z represents Ile or Leu.

2. A polypeptide according to claim 1, wherein the proportion (molar ratio) of the peptide unit (1) relative to the peptide unit (2) is 99/1 to 1/99.

3. A polypeptide according to claim 1, wherein
(i) X is Hyp,
Y is Gln, Asn, Leu, Ile, Val or Ala, and
Z is Ile or Leu; or
(ii) X is Pro,
Y is Gln, Asn, Leu, Ile, Val or Ala, and
Z is Ile or Leu.

4. A polypeptide according to claim 1, which is degradable with a collagenase.

5. A polypeptide according to claim 1, which shows positive Cotton effect at a wavelength in range of 220 to 230 nm and negative Cotton effect at a wavelength in range of 195 to 205 nm in a circular dichroism spectrum, and wherein at least part of the polypeptide is capable of forming a triple helical structure.

6. A polypeptide according to claim 1, which shows a peak of the molecular weight in the range from 5×10² to 500×10⁴ in the molecular weight distribution.

7. A polypeptide according to claim 1, which is capable of forming a collagenous tissue.

8. A process for producing a polypeptide recited in claim 1, which comprises condensing an amino acid component or peptide component which at least contains an amino acid or peptide fragment corresponding to the formula (1) recited in claim 1 and an amino acid or peptide fragment corresponding to the formula (2) recited in claim 1.

9. A process for producing a polypeptide recited in claim 1, which comprises
(a) condensing a peptide component which at least contains a peptide having the both amino acid sequences represented by the formulae (1) and (2) recited in claim 1, respectively; or
(b) condensing a peptide component which at least contains a peptide having an amino acid sequence represented by the formula (1) and a peptide having an amino acid sequence represented by the formula (2).

10. A process according to claim 8 or 9, wherein the condensation step is carried out in the presence of (i) a dehydrating and condensing agent, or (ii) a dehydrating and condensing agent and a condensing auxiliary.

11. A process according to claim 10, wherein the dehydrating and condensing agent comprises at least one member selected from the group consisting of a carbodiimide-series condensing agent, a fluorophosphate-series condensing agent, and a diphenylphosphorylazide.

12. A process according to claim 10, wherein the condensation step is carried out in the presence of a non-aqueous solvent, and the proportion of the dehydrating and condensing agent is 0.7 to 5 mol relative to 1 mol of the total amount of the amino acid component or peptide component.

13. A process according to claim 10, wherein the condensation step is carried out in the presence of an aqueous solvent, and the proportion of the dehydrating and condensing agent is 2 to 500 mol relative to 1 mol of the total amount of the amino acid component or peptide component.

14. A process according to claim 10, wherein the condensing auxiliary comprises at least one member selected from the group consisting of an N-hydroxypolycarboxylic acid imide, an N-hydroxytriazole, a triazine, and ethyl ester of 2-hydroxyimino-2-cyanoacetic acid.

15. A process according to claim 10, wherein the proportion of the condensing auxiliary is 0.5 to 5 mol relative to 1 mol of the total amount of the amino acid component or the peptide component.

16. A collagenase inhibitor comprising a polypeptide recited in claim 1.

17. A cosmetic preparation comprising a polypeptide recited in claim 1, and inhibiting collagenase activity.

18. A food composition comprising a polypeptide recited in claim 1, and inhibiting collagenase activity.

19. A method for inhibiting a collagenase activity which comprises acting a polypeptide recited in claim 1 on a collagenase.
